(19) United States
(12) Patent Application Publication
(10) Pub. No.: US 2019/0077744 A1
(43) Pub. Date: Mar. 14, 2019

(54) METHOD FOR COUPLING A FIRST COMPOUND TO A SECOND COMPOUND

(71) Applicant: Dow Global Technologies LLC, Midland, MI (US)

(72) Inventors: Patrick S. Hanley, Midland, MI (US); Thomas P. Clark, Midland, MI (US); Arkady L. Krasovskiy, Lake Jackson, TX (US); Matthias S. Ober, Midland, MI (US)

(21) Appl. No.: 16/085,035

(22) PCT Filed: Mar. 2, 2017
(86) PCT No.: PCT/US2017/020427

§ 371 Date: Sep. 14, 2018

Related U.S. Application Data
(60) Provisional application No. 62/308,364, filed on Mar. 15, 2016.

(51) Int. Cl. C07C 209/66; C07C 209/22

(52) U.S. Cl. CPC C07C 209/66; C07C 211/55; C07C 209/22

(57) ABSTRACT

The present disclosure describes a method of coupling a first compound to a second compound, the method comprising: providing the first compound having a fluorosulfonate substituent; providing the second compound comprising an amine; and reacting the first compound and the second compound in a reaction mixture, the reaction mixture including a catalyst having at least one group 10 atom, the reaction mixture including a base, the base comprising a carbonate salt, a phosphate salt or an acetate salt, the reaction mixture under conditions effective to couple the first compound to the second compound. The present disclosure further describes a one-pot method for coupling a first compound to a second compound in the presence of a mild base.

### BACKGROUND

Buchwald-Hartwig coupling (C—N coupling) is a valuable synthetic method for coupling compounds, thereby forming a new carbon-nitrogen bond between a first compound and a second compound. Traditionally, C—N coupling partners consist of a first compound having a halide or sulfonate substituent and a second compound comprising an amine. It is common for the first compound to comprise an aryl compound.

It is known that triflates (trifluoromethanesulfonate), having the formula F₃CSO₂—, may be used in the place of the halides in C—N couplings, however the expense of triflic anhydride (CF₃SO₂)₂O has limited the use of triflates in C—N couplings to the production of fine chemicals. Further, the atom economy of triflic anhydride is low since half of the molecule is expended as monomeric triflate anion (CF₃SO₂⁻) as a result of condensation with a phenolic precursor.

It is also known that aryl methanesulfonates (also known as mesylates) are suitable for aryl-amine cross-coupling reactions. One drawback of aryl-amine crosscouping using aryl methanesulfonates is that these reactions require expensive palladium catalysts. Another drawback of aryl-amine cross-coupling reactions using aryl methanesulfonates is low atom economy.

When performing a C—N coupling using either a triflate or methanesulfonate, it is common to use harsh reaction conditions. These harsh reaction conditions are necessary when using the typical leaving groups, for example triflate or methanesulfonate, to achieve adequate conversion and yield. Furthermore, such harsh reaction conditions are necessary to accomplish suitable fast reactions. Mild bases are typically unsuitable for use with the leaving groups commonly used in C—N couplings.

Cross-coupling reactions performed in mild conditions are desired.

### STATEMENT OF INVENTION

The present disclosure describes a method of coupling a first compound to a second compound, the method comprising: providing the first compound having a fluorosulfonate substituent; providing the second compound comprising an amine; and reacting the first compound and the second compound in a reaction mixture, the reaction mixture including a catalyst having at least one group 10 atom, the reaction mixture including a base, the base comprising a carbonate salt, a phosphate salt or an acetate salt, the reaction mixture under conditions effective to couple the first compound to the second compound.

The present disclosure describes a method for coupling a first compound A¹ a second compound A², as illustrated in Equation 1, comprising:

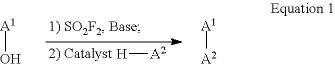

providing the first compound A¹ having a hydroxyl substituent, sulfuryl fluoride and a base to a reaction mixture, the first compound A¹ comprising an aryl group or a heteroaryl group, the base comprising a carbonate salt, a phosphate salt or an acetate salt;

providing a catalyst comprising a group 10 atom and the second compound A² to the reaction mixture, the second compound A² as defined in Equation 4:

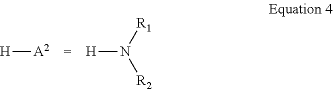

the second compound A² comprising an amine wherein each of R¹ and R² are independently Hydrogen, an aryl group, a heteroaryl group, an alkyl group, a cycloalkyl group, a nitro group, a halide, a nitrogen, a cyano group, a carboxyester group, an acetoxy group, a substituted alkyl, aryl, heteroaryl or cycloalkyl group, or R¹ and R² are constituent parts of a ring system;

reacting the first compound and the second compound under conditions effective to couple the first compound to the second compound.

### DETAILED DESCRIPTION

Unless otherwise indicated, numeric ranges, for instance “from 2 to 10,” are inclusive of the numbers defining the range (e.g., 2 and 10).

Unless otherwise indicated, ratios, percentages, parts, and the like are by moles.

As used herein, unless otherwise indicated, the phrase “molecular weight” refers to the number average molecular weight as measured in the conventional manner.

“Alkyl,” as used in this specification, whether alone or as part of another group (e.g., in dialkylamino), encompasses straight and branched chain aliphatic groups having the indicated number of carbon atoms. If no number is indicated (e.g., aryl-alkyl-), then 1-12 alkyl carbons are contemplated. Preferred alkyl groups include, without limitation, methyl, ethyl, propyl, isopropyl, butyl, isobutyl, sec-butyl, tert-butyl, pentyl, hexyl and tert-octyl.

The term “heteroalkyl” refers to an alkyl group as defined above with one or more heteroatoms (nitrogen, oxygen, sulfur, phosphorus) replacing one or more carbon atoms within the radical, for example, an ether or a thioether.

An “aryl” group refers to any functional group or substituent derived from an aromatic ring. In one instance, aryl refers to an aromatic moiety comprising one or more aromatic rings. In one instance, the aryl group is a C₆-C₁₈ aryl group. In one instance, the aryl group is a C₆-C₁₀ aryl group. In one instance, the aryl group is a C₁₀-C₁₈ aryl group. Aryl groups contain 4n+2 pi electrons, where n is an integer. The aryl ring may be fused or otherwise attached to one or more heteroaryl rings, aromatic or non-aromatic hydrocarbon rings or heterocycloalkyl rings. Preferred aryls include, without limitation, phenyl, naphthyl, anthracenyl, and fluorenyl. Unless otherwise indicated, the aryl group is optionally substituted with 1 or more substituents that are compatible with the syntheses described herein. Such substituents include, but are not limited to, sulfonate groups, boron-containing groups, alkyl groups, nitro groups, halogens, cyano groups, carboxylic acids, esters, amides, C₂-C₈ alkene, and other aromatic groups. Other substituents are known in the art. Unless otherwise indicated, the foregoing substituent groups are not themselves further substituted.

“Heteroaryl” refers to any functional group or substituent derived from an aromatic ring and containing at least one heteroatom selected from nitrogen, oxygen, and sulfur. Preferably, the heteroaryl group is a five or six-membered ring. The heteroaryl ring may be fused or otherwise attached to one or more heteroaryl rings, aromatic or non-aromatic hydrocarbon rings or heterocycloalkyl rings. Examples of heteroaryl groups include, without limitation, pyridine, pyrimidine, pyridazine, pyrrole, triazine, imidazole, triazole, furan, thiophene, oxazole, thiazole. The heteroaryl group may be optionally substituted with one or more substituents that are compatible with the syntheses described herein. Such substituents include, but are not limited to, fluorosulfonate groups, boron-containing groups, C₁-C₈ alkyl groups, nitro groups, halogens, cyano groups, carboxylic acids, esters, amides, C₂-C₈ alkene and other aromatic groups. Other substituents are known in the art. Unless otherwise indicated, the foregoing substituent groups are not themselves further substituted.

“Aromatic compound” refers to a ring system having 4n+2 pi electrons where n is an integer.

As noted above, the present disclosure describes a process for coupling a first compound to a second compound. This process is shown generally in Equation 1, whereby a first compound having a hydroxyl group is first reacted with SO₂F₂ and a base and is second reacted with a second compound comprising an amine in the presence of a catalyst. It is understood that where a hydroxyl group is indicated, the hydroxyl group could be deprotonated to form a phenolate (e.g. the deprotonation step could be performed prior to introduction of A¹ to the reaction mixture or after the introduction to the reaction mixture).

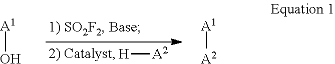

Unexpectedly, it has been found that the reaction of Equation 1 may be performed as a one-pot reaction, as compared to performing the reaction in discrete steps. Without being limited by theory, it is anticipated that the reaction shown in Equation 1 proceeds along the same reaction path whether performed as a one-pot reaction or as discrete steps. When performed in discrete steps, the first step comprises reacting a first compound having a hydroxyl substituent with SO₂F₂ to yield the product shown in Equation 2, and the second step comprises reacting the product of Equation 2 with a second compound comprising an amine to yield the product shown in Equation 3.

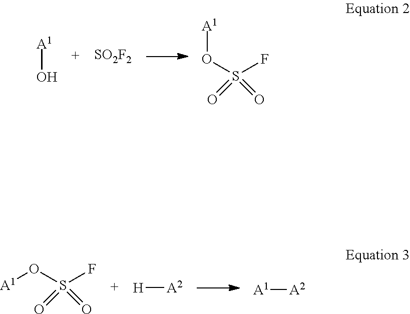

In one instance, the process involves a one-pot reaction where a first compound having a hydroxyl group is first reacted with SO₂F₂ and a base and is second reacted with a second compound comprising an amine in the presence of a catalyst, as shown generally in Equation 1. Without being limited by theory, it is expected that Equation 3 is the same general reaction as depicted by step 2) of the reaction shown in Equation 1.

As used in Equation 1, Equation 2 and Equation 3, the first compound is identified as A¹. The first compound is either an aryl group or a heteroaryl group. The second compound is identified as A² as illustrated in Equation 4:

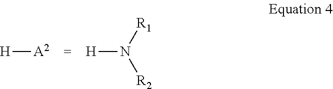

The second compound A² is an amine wherein R¹ and R² are each independently H or other suitable substituent suitable for use in a C—N coupling. In one instance, R¹ and R² are each independently H, alkyl or aryl groups. The result of the reactions shown in Equation 1 and Equation 3 is the formation of a new carbon-nitrogen bond between the first compound and the second compound, thereby coupling the first compound to the second compound.

As noted above in the first step of Equation 1 and in Equation 2, the first compound is bonded to a fluorosulfonate group. A fluorosulfonate group refers to O-fluorosulfonate of the formula —OSO₂F. O-fluorosulfonate may be synthesized from sulfuryl fluoride. The fluorosulfonate group serves as a leaving group from the first compound. Without being limited by theory, the sulfur atom of the fluorosulfonate group is bonded to the oxygen of the hydroxyl group of the first compound.

As noted above, the second compound is an amine. The amine is alternatively ammonia, a primary or a secondary amine R¹ and R² are each independently a substituent suitable for use in a C—N coupling, for example, Hydrogen, aryl, heteroaryl, alkyl, heteroakyl, amide, carbonaryl, carbonheteroaryl, halide, Nitrogen, carbonyl or acetoxy. In one instance, the amine includes an R¹ and R² that are members of one or more rings, for example, a cyclic amine, a di-alkyl amine or di-aryl amine. In one instance, R¹ and R² are bonded to each other. In one instance, each of R¹ and R² are independently C₁₋₁₈ alkyl, C₃₋₁₈ cycloalkyl, C₆₋₁₈ aryl, or H. In one instance, the alkyl or aryl groups of the amine are themselves further substituted.

As noted above in Equation 1 and Equation 3, the first compound is reacted with the second compound in a reaction mixture. The reaction mixture includes a catalyst having at least one group 10 atom. In some instances, the reaction mixture also includes a ligand, and a base. The group 10 atoms include nickel, palladium and platinum.

The catalyst is provided in a form suitable to the reaction conditions. In one instance, the catalyst is provided on a substrate. In one instance, the catalyst having at least one group 10 atom is generated in situ from one or more precatalysts and one or more ligands. Examples of palladium precatalysts include, but are not limited to, Palladium(II) acetate, Palladium(II) chloride, Dichlorobis(acetonitrile)palladium(II), Dichlorobis(benzonitrile)palladium(II), Allylpalladium chloride dimer, Palladium(II) acetylacetonate, Palladium(II) bromideBis(dibenzylideneacetone)palladium(0), Bis(2-methylallyl)palladium chloride dimer, Crotylpalladium chloride dimer, Dichloro(1,5-cyclooctadiene)palladium(II), Dichloro(norbornadiene)palladium(II), Palladium(II) trifluoroacetate, Palladium(II) benzoate, Palladium(II) trimethylacetate, Palladium(II) oxide, Palladium(II) cyanide, Tris(dibenzylideneacetone)dipalladium(0), Palladium(II) hexafluoroacetylacetonate, cis-Dichloro(N,N,N′,N′-tetramethylethylenediamine) palladium(II), Cyclopentadienyl[(1,2,3-n)-1-phenyl-2-propenyl]palladium(II), and Allyl(cyclopentadienyl)palladium(II).

In one instance, nickel-based catalysts are used. In another instance, platinum-based catalysts are used. In yet another instance, a catalyst including one or more of nickel, platinum and palladium-based catalysts are used.

In one instance, pyridine-enhanced precatalyst preparation stabilization and initiation (PEPPSI) type catalysts are used, for example, [1,3-Bis(2,6-Diisopropylphenyl)imidazol-2-ylidene](3-chloropyridyl)palladium(II) dichloride, and (1,3-Bis(2,6-diisopropylphenyl)imidazolidene) (3-chloropyridyl) palladium(II) dichloride.

Examples of nickel precatalysts include, but are not limited to, nickel(II) acetate, nickel(II) chloride, Bis(triphenylphosphine)nickel(II) dichloride, Bis(tricyclohexylphosphine)nickel(II) dichloride, [1,1′-Bis(diphenylphosphino)ferrocene]dichloronickel(II), Dichloro[1,2-bis(diethylphosphino)ethane]nickel(II), Chloro(1-naphthyl)bis(triphenylphosphine)nickel(II), 1,3-Bis(2,6-diisopropylphenyl)imidazolium chloride, Bis(1,5-cyclooctadiene)nickel(0), Nickel(II) chloride ethylene glycol dimethyl ether complex, [1,3-Bis(diphenylphosphino)propane]dichloronickel(II), [1,2-Bis(diphenylphosphino)ethane]dichloronickel(II), and Bis(tricyclohexylphosphine)nickel(0).

The ligand used in the reaction mixture is preferably selected to generate the selected catalyst from a pre-catalyst. For example, the ligand may be a phosphine ligand, a carbene ligand, an amine-based ligand, a carboxylate based ligand, an aminodextran, an aminophosphine-based ligands or an N-heterocyclic carbene-based ligand. In one instance, the ligand is monodentate. In one instance, the ligand is bidentate. In one instance, the ligand is polydentate.

Suitable phosphine ligands may include, but are not limited to, mono- and bi-dentate phosphines containing functionalized aryl or alkyl substituents or their salts. For example, suitable phosphine ligands include, but are not limited to, triphenylphosphine; Tri(o-tolyl)phosphine; Tris(4-methoxyphenyl)phosphine; Tris(pentafluorophenyl)phosphine; Tri(p-tolyl)phosphine; Tri(2-furyl)phosphine; Tris(4-chlorophenyl)phosphine; Di(1-adamantyl)(1-naphthoyl)phosphine; Benzyldiphenylphosphine; 1,1′-Bis(di-t-butylphosphino)ferrocene; (−)-1,2-Bis((2R,5R)-2,5-dimethylphospholano)benzene; (−)-2,3-Bis[(2R,5R)-2,5-dimethylphospholanyl]-1-[3,5-bis(trifluoromethyl)phenyl]-1H-pyrrole-2,5-dione; 1,2-Bis(diphenylphosphino)benzene; 2,2′-Bis(diphenylphosphino)-1,1′-binaphthyl; 2,2′-Bis(diphenylphosphino)-1,1′-biphenyl, 1,4-Bis(diphenylphosphino)butane; 1,2-Bis(diphenylphosphino)ethane; 2-[Bis(diphenylphosphino)methyl]pyridine; 1,5-Bis(diphenylphosphino)pentane; 1,3-Bis(diphenylphosphino)propane; 1,1′-Bis(di-i-propylphosphino)ferrocene; (S)-(−)-5,5′-Bis[di(3,5-xylyl)phosphino]-4,4′-bi-1,3-benzodioxole; tricyclohexylphosphine (referred to herein as PCy3); Tricyclohexylphosphine tetrafluoroborate (referred to herein as PCy3.HBF₄); N-[2-(di-1-adamantylphosphino) phenyl]morpholine; 2-(Di-t-butylphosphino)biphenyl; 2-(Di-t-butylphosphino)-3,6-dimethoxy-2′,4′,6′-tri-i-propyl-1,1′-biphenyl; 2-Di-t-butylphosphino-2′-(N,N-dimethylamino)biphenyl; 2-Di-t-butylphosphino-2′-methylbiphenyl; Dicyclohexylphenylphosphine; 2-(Dicyclohexylphosphino)-3,6-dimethoxy-2′,4′,6′-tri-i-propyl-1; 2-(Dicyclohexylphosphino)-2′-(N,N-dimethylamino)biphenyl; 2-Dicyclohexylphosphino-2′,6′-dimethylamino-1,1′-biphenyl; 2-Dicyclohexylphosphino-2′,6′-di-i-propoxy-1,1′-biphenyl; 2-Dicyclohexylphosphino-2′-methylbiphenyl; 2-[2-(Dicyclohexylphosphino)phenyl]-1-methyl-1H-indole; 2-(Dicyclohexylphosphino)-2′,4′,6′-tri-i-propyl-1,1′-biphenyl; [4-(N,N-Dimethylamino)phenyl]di-t-butylphosphine; 9,9-Dimethyl-4,5-bis(diphenylphosphino)xanthene; (R)-(−)-1-[(S)-2-(Diphenylphosphino)ferrocenyl]ethyldicyclohexylphosphine; Tribenzylphosphine; Tri-t-butylphosphine; Tri-n-butylphosphine; and 1,1′-Bis(diphenylphosphino)ferrocene (referred to herein as “DPPF”).

Suitable amine and aminophosphine-based ligands include any combination of monodentate or bidentate alkyl and aromatic amines including, but not limited to, pyridine, 2,2′-Bipyridyl, 4,4′-Dimethyl-2,2′-dipyridyl, 1,10-Phenanthroline, 3,4,7,8-Tetramethyl-1,10-phenanthroline, 4,7-Dimethoxy-1,10-phenanthroline, N,N,N′,N′-Tetramethylethylenediamine, 1,3-Diaminopropane, ammonia, 4-(Aminomethyl)pyridine, (1R,2S,9S)-(+)-11-Methyl-7,11-diazatricyclo[7.3.1.0^{2,7}]tridecane, 2,6-Di-tert-butylpyridine, 2,2′-Bis[(4S)-4-benzyl-2-oxazoline], 2,2-Bis((4S)-(−)-4-isopropyloxazoline)propane, 2,2′-Methylenebis[(4S)-4-phenyl-2-oxazoline], and 4,4′-di-tert-butyl-2,2′bipyridyl. In addition, aminophosphine ligands such as 2-(Diphenylphosphino)ethylamine, 2-(2-(Diphenylphosphino)ethyl)pyridine, (1R,2R)-2-(diphenylphosphino)cyclohexanamine, an aminodextran and 2-(Di-tert-butylphosphino)ethylamine.

Suitable carbene ligands include N-heterocyclic carbene (NHC) based ligands, including, but not limited to, 1,3-Bis(2,4,6-trimethylphenyl)imidazolinium chloride, 1,3-Bis(2,6-diisopropylphenyl)imidazolium chloride, 1,3-Bis-(2,6-diisopropylphenyl) imidazolinium chloride, 1,3-Diisopropylimidazolium chloride, and 1,3-Dicyclohexylbenzimidazolium chloride.

The base used in the reaction mixture is selected to be compatible with the catalyst, the amine and the fluorosulfonate. Unexpectedly, it has been found that mild bases that would otherwise provide low yields and/or slow kinetics when used with other leaving groups are suitable for use when the leaving groups is a fluorosulfonate substituent. Suitable bases include, but are not limited to, carbonate salts, phosphate salts, acetate salts and carboxylic acid salts. Inorganic bases are suitable in the reaction mixture. As used herein, “inorganic base” refers to non-organic bases, for example, carbonate salts, phosphate salts, and acetate salts.

Examples of carbonate salts include, but are not limited to, lithium carbonate, sodium carbonate, potassium carbonate, rubidium carbonate, cesium carbonate, ammonium carbonate, substituted ammonium carbonates, and the corresponding hydrogen carbonate salts. Examples of phosphate salts include, but are not limited to, lithium phosphate, sodium phosphate, potassium phosphate, rubidium phosphate, cesium phosphate, ammonium phosphate, substituted ammonium phosphates, and the corresponding hydrogen phosphate salts. Examples of acetate salts include, but are not limited to, lithium acetate, sodium acetate, potassium acetate, rubidium acetate, cesium acetate, ammonium acetate, and substituted ammonium acetates.

In one instance, the base is used in the presence of a phase-transfer catalyst. In another instance, the base is used in the presence of water. In yet another instance, the base is used in the presence of an organic solvent. In still another instance, the base is used in the presence of one or more of a phase-transfer catalyst, water or an organic solvent.

Preferably, at least one equivalent of base is present for each equivalent of fluorosulfonate. In some embodiments, no more than 10 equivalents of base are present for each equivalent of fluorosulfonate. In some embodiments, at least 2 equivalents of base are present for each equivalent of fluorosulfonate. In some embodiments, no more than 6 equivalents of base are present for each equivalent of fluorosulfonate.

The solvent in the reaction mixture is selected such that it is suitable for use with the reactants, the catalyst, the ligand and the base. For example, suitable solvents include toluene, xylenes (ortho-xylene, meta-xylene, para-xylene or mixtures thereof), benzene, water, methanol, ethanol, 1-propanol, 2-propanol, n-butanol, 2-butanol, pentanol, hexanol, tert-butyl alcohol, tert-amyl alcohol, ethylene glycol, 1,2-propanedioal, 1,3-propanediol, glycerol, N-methyl-2-pyrrolidone, acetonitrile, N,N-dimethylformamide, methyl acetate, ethyl acetate, propyl acetate, isopropyl acetate, triacetin, acetone, methyl ethyl ketone, and ethereal solvents, such as 1,4-dioxane, tetrahydrofuran, 2-methyltetrahydrofuran, diethylether, cyclopenyl methyl ether, 2-butyl ethyl ether, dimethoxyethane, polyethyleneglycol. In one instance, the solvent includes any combination of the solvents described herein, in, or in the absence of, a surfactant. In one instance, the sulfuryl fluoride is used neat at a sufficiently low temperature that the sulfuryl fluoride is in a liquid. In one instance, water is included in the reaction mixture.

One benefit of using fluorosulfonates as compared to triflates, is that the reaction can be carried out without a subsequent separation step, or with a simple separation step. In couplings involving triflates, a dedicated purification step is required to remove byproducts since the products and the byproducts typically occupy the same phase. In the reaction schemes described herein, the byproducts are either in the gas phase, and will bubble out spontaneously or with a simple degassing step, or will partition into the aqueous phase, which is easily separable. As such, the reaction scheme described herein provides additional benefits as compared to C—N couplings involving triflates.

In one instance, the reaction described herein is completed as a one-pot reaction as shown in Equation 1. In a first step, an compound having an alcohol substituent is added to a reaction mixture in the presence of sulfuryl fluoride and a base. The base may be any of the bases described herein, including, without limitation, amine bases and inorganic bases. This first step couples the fluorosulfonate substituent to the oxygen of the hydroxyl group. To the reaction mixture formed during this first step is added a second compound comprising an amine and a catalyst. The catalyst may be a suitable group 10 catalyst, including, without limitation, platinum, palladium and nickel catalysts. The product of this second step is a compound formed by coupling the first compound and the second compound.

Some embodiments of the invention will now be described in detail in the following Examples. Unless stated otherwise, reported yields are ±5%.

### Example

A series of reactions are described where an aryl electrophile is reacted with phenylamine as shown in Equation 5, where X is a leaving group listed in Table 1.

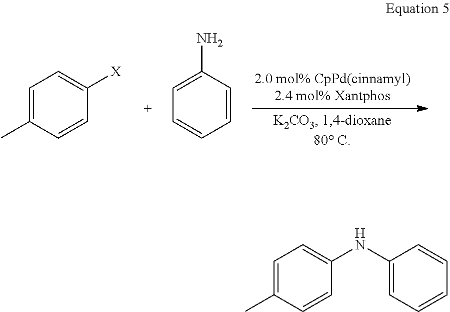

The reactions of the present Example are performed in a nitrogen-purged glovebox. 7 30 mL glass vials are provided as reaction vessels. To each vial is added the aryl electrophile identified in Table 1 (0.5 mmol), Xantphos (0.313 mL as 0.0384 M solution in 1,4-dioxane; 0.012 mmol); potassium carbonate (0.138 g; 1.00 mmol), and 1,4-dioxane (5 mL). To the stirring mixture is added phenylamine (0.327 mL as 1.835 M solution in 1,4-dioxane; 0.6 mmol), CpPd(cinnamyl) (0.270 mL as 0.037 M solution in 1,4-dioxane; 0.01 mmol) and 1,3,5-trimethoxybenzene (0.05 mmol) as an internal standard. The reaction mixture is heated to 80° C. for 24 hours. After 7 hours a ˜0.5 mL aliquot is taken for GCMS and ¹H NMR analysis. After 24 hours the reaction mixture is cooled to room temperature and 1,4-dioxane is gently evaporated at reduced pressure and the residue is analyzed by GCMS and ¹H NMR. The yield and conversion are calculated based on relative integrals of 1,3,5-trimethoxybenzene (internal standard) vs. peaks of starting material and product, as reported in Table 1.

TABLE 1

| | | Conversion | Conversion | |
|---|---|---|---|---|
| Reac- | | Ar—X | Ar—X | Yield^{b} |
| tion | X | (%) 7 h | (%) 24 h | (%) (24 h) |
| 1 | —OSO₂F (OFs) | 95 | 100 | 96 |
| 2 | —OSO₂CF₃ (OTf) | 20 | 92 | 84 |
| 3 | Cl | 10 | 27 | 13 |
| 4 | Br | 2 | 16 | 6 |
| 5 | I | 0 | 2 | <2 |
| 6 | —OSO₂CH₃ (OMs) | 0 | 0 | 0 |
| 7 | —OSO₂₋(4- | 0 | 0 | 0 |
| | CH₃C₆H₄) (OTs) | | | |

In industrial chemistry, yield, conversion and reaction rate are important considerations when designing a reaction pathway. As is shown in the Example, when using a mild base in the reaction scheme described herein, the fluorosulfonate leaving group provides the best yield and conversion within twenty four hours, and remarkably better conversion within seven hours. The methods described herein provides 80 percent or greater conversion of the first compound having a fluorosulfonate substituent within seven hours. The methods described herein provides 90 percent or greater conversion of the first compound having a fluorosulfonate substituent within seven hours. The methods described herein provides 95 percent or greater conversion of the first compound having a fluorosulfonate substituent within seven hours. The methods described herein provides 80 percent or greater conversion of the first compound having a fluorosulfonate substituent within twenty four hours. The methods described herein provides 90 percent or greater conversion of the first compound having a fluorosulfonate substituent within twenty four hours. The methods described herein provides 95 percent or greater conversion of the first compound having a fluorosulfonate substituent within twenty four hours. The methods described herein provides 80 percent or greater yield of the target reaction product within twenty four hours. The methods described herein provides 90 percent or greater conversion of the target reaction product within twenty four hours. The methods described herein provides 95 percent or greater conversion of the target reaction product within twenty four hours.

1. A method of coupling a first compound to a second compound, method comprising:
providing the first compound having a fluorosulfonate substituent, the first compound comprising an aryl or a heteroaryl group;
providing the second compound comprising an amine; and
reacting the first compound and the second compound in a reaction mixture, the reaction mixture including a catalyst having at least one group 10 atom, the reaction mixture including a base, the base comprising a carbonate salt, a phosphate salt or an acetate salt, the reaction mixture under conditions effective to couple the first compound to the second compound.

2. The method of claim 1, wherein the reaction mixture further includes a ligand.

3. The method of claim 1 wherein the catalyst is generated in-situ from a palladium precatalyst.

4. The method of claim 1 wherein the catalyst is generated in-situ from a palladium precatalyst, the palladium precatalyst is selected from the group consisting of: Palladium(II) acetate, Palladium(II) chloride, Dichlorobis(acetonitrile)palladium(II), Dichlorobis(benzonitrile)palladium(I1), Allylpalladium chloride dimer, Palladium(II) acetylacetonate, Palladium(II) bromideBis(dibenzylideneacetone)palladium(0), Bis(2-methylallyl)palladium chloride dimer, Crotylpalladium chloride dimer, Dichloro(1,5-cyclooctadiene)palladium(II), Dichloro(norbornadiene)palladium(II), Palladium(II) trifluoroacetate, Palladium(II) benzoate, Palladium(II) trimethylacetate, Palladium(II) oxide, Palladium(II) cyanide, Tris(dibenzylideneacetone)dipalladium(0), Palladium(II) hexafluoroacetylacetonate, cis-Di chloro(N,N,N,N′-tetramethylethylenediamine)palladium(II), and Cyclopentadienyl[(1,2,3-n)-1-phenyl-2-propenyl]palladium(II), Allyl(cyclopentadienyl)paladium(II), [1,3-Bis(2,6-Diisopropyphenyl)imidazol-2-ylidene](3-chloropyridyl)palladium(II)dichloride, and (1,3-Bis(2,6-diisopropylphenyl)imidazolidene) (3-chloropyridyl) palladium(II) dichloride, and a mixture of two or more thereof.

5. The method of claim 1 wherein the ligand is a phosphine ligand or a carbene ligand.

6. The method of claim 1 wherein the ligand is an amine-based ligand, an aminophosphine-based ligand, an N-heterocyclic carbene-based ligand, a monodentate or bidentate alkyl amine, or a monodentate or bidentate aromatic amine.

7. The method of claim 1, wherein the base is selected from the group consisting of lithium carbonate, sodium carbonate, potassium carbonate, rubidium carbonate, cesium carbonate, ammonium carbonate, substituted ammonium carbonates, hydrogen carbonates, lithium phosphate, sodium phosphate, potassium phosphate, rubidium phosphate, cesium phosphate, ammonium phosphate, substituted ammonium phosphates, hydrogen phosphates, lithium acetate, sodium acetate, potassium acetate, rubidium acetate, cesium acetate, ammonium acetate, substituted ammonium acetates, and a mixture of two or more thereof.

8. The method of claim 1, wherein the reaction mixture includes a solvent.

9. The method of claim 8, wherein the solvent is selected from the group consisting of toluene, xylene, benzene, chlorobenzene, water, methanol, ethanol, 1-propanol, 2-propanol, n-butanol, 2-butanol, pentanol, hexanol, tert-butyl alcohol, tert-amyl alcohol, ethylene glycol, 1,2-propanedioal, 1,3-propanediol, glycerol, N-methyl-2-pyrrolidone, acetonitrile, N,N-dimethylformamide, methyl acetate, ethyl acetate, propyl acetate, isopropyl acetate, triacetin, acetone, methyl ethyl ketone, and ethereal solvents, such as 1,4-dioxane, tetrahydrofuran, 2-methyltetrahydrofuran, diethylether, cyclopenyl methyl ether, 2-butyl ethyl ether, dimethoxyethane, polyethyleneglycol, Dimethylacetamide(DMA), dimethylsulfoxide (DMSO), and 1,2-dichloroethane (DCE).

10. The method of claim 1, wherein the reaction mixture includes water.

11. The method of claim 1, wherein the method provides 80 percent or greater conversion of the first compound having a fluorosulfonate substituent at seven hours.

12. The method of claim 1, wherein the method provides 80 percent or greater yield within twenty four hours.

13. A method for coupling a first compound A¹ a second compound A², as illustrated in Equation 1, comprising: 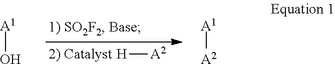
providing the first compound A¹ having a hydroxyl substituent, sulfuryl fluoride and a base to a reaction mixture, the first compound A¹ comprising an aryl group or a heteroaryl group, the base comprising a carbonate salt, a phosphate salt or an acetate salt;
providing a catalyst comprising a group 10 atom and the second compound A² to the reaction mixture, the second compound A² as defined in Equation 4: 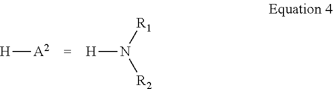
the second compound A² comprising an amine wherein each of R¹ and R² are independently Hydrogen, an aryl group, a heteroaryl group, an alkyl group, a cycloalkyl group, a nitro group, a halide, a nitrogen, a cyano group, a carboxyester group, an acetoxy group, a substituted alkyl, aryl, heteroaryl or cycloalkyl group, or R¹ and R² are constituent parts of a ring system;
reacting the first compound and the second compound under conditions effective to couple the first compound to the second compound.

14. The method of claim 13, wherein the reaction mixture further includes a ligand.

15. The method of claim 13 wherein the catalyst is generated in-situ from a palladium precatalyst.

16. The method of claim 13 wherein the ligand is a phosphine ligand or a carbene ligand.

17. The method of claim 13 wherein the ligand is an amine-based ligand, an aminophosphine-based ligand or an N-heterocyclic carbene-based ligand.

18. The method of claim 13 wherein the ligand is a monodentate or bidentate alkyl amine or a monodentate or bidentate aromatic amine.

19. The method of claim 13, wherein the reaction mixture includes a solvent.
